(19) United States
(12) Patent Application Publication
(10) Pub. No.: US 2005/0015016 A1
(43) Pub. Date: Jan. 20, 2005

(54) Stress probe for a vehicle operator

(76) Inventors: Ercan Elitok, Ulm (DE); Stefan Hahn, Ulm (DE); Wilhelm Kincses, Esslingen (DE); Michael Schrauf, Langenfeld (DE)

Correspondence Address: PENDORF & CUTLIFF, 5111 MEMORIAL HIGHWAY, TAMPA, FL, 33634-7356, US

(21) Appl. No.: 10/891,222

(22) Filed: Jul. 14, 2004

(30) Foreign Application Priority Data
Jul. 14, 2003 (DE) 103 31 800.3

(51) Int. Cl. A61B 5/04

(52) U.S. Cl. 600/544; 340/576

(57) ABSTRACT

A process and system for monitoring the ability to work under pressure or receptiveness and effectiveness of a vehicle operator. The EEG-patterns of the mental activity are continuously recorded in various frequency ranges and evaluated according to various criteria. The occurrence of signals shapes or changes which indicate a impairment of operating ability are thus detected and lead, depending upon the detected status, to triggering of stepwise gradually increasing responses, from warning signals up to direct intervention and the control of the vehicle.

### BACKGROUND OF THE INVENTION

1. Field of the Invention

The invention concerns process and devices for detecting situation-dependent physiological and mental states of a person. The invention in particular concerns systems for monitoring the workload stress level, receptiveness and effectiveness of a person during operation of a vehicle.

2. Related Art of the Invention

Various demands are placed upon the driver while operating a vehicle. Depending upon the operating situation (for example traffic density, weather factors), secondary tasks (for example navigation, receiving traffic reports, distraction by passengers) and driver characteristics (age, experience) these demands, in combination with the mental state of the driver (for example, fatigue) produce various levels of ability to work under pressure. The objective recognition of the actual workload stress level and mental condition of the driver is of significant importance in improving the human-machine interaction and is thus useful for general traffic safety.

Generally, the operating effectiveness of a person at various stages of work-loading is determined by the accomplishment of, or ability to cope with, secondary tasks using a questionnaire. This process is, in principle, strongly error-prone, since the weighting of the tasks during accomplishment of the questionnaire is strongly differentiated among different individuals. Besides this, the methodology of the secondary task is always inherently associated with interference with the main task (problem of the association of the results). In general, the process does not correlate well with the above described “ad hoc” load situation during operating of a vehicle.

Another process uses the variability of brain activity occurring during varying activity for surveying the actual mental condition of a person. One system for analysis of data regarding the condition and alertness of a vehicle operator based upon this principle is described in U.S. Pat. No. 5,884,626 (Kuroda, et al.). In this system fluctuations of special brain waves are recorded at a certain frequency band using an electroencephalogram (EEG) and analyzed for significant changes. Depending upon the objective (alertness/fatigue probe), here only frequency segments in the range of 8 to 13 hertz (corresponding to the so-called α-band) are detected, since in this frequency intervals of tiredness produce particularly noticeable signal changes.

This technique is associated with various disadvantages and flaws. On the one hand, using this predetermined frequency band principle is it not possible to obtain a time resolution of less than 100 ms (=duration of a 10 Hz sinusoidal oscillation). Therewith, various other possible influences on the actual load condition cannot be detected. The signal activity in the α-band provides, besides this, only a general value for the overall condition of the alertness (=low α-activity) or as the case may be tiredness (=high α-activity) of a person. With this process it is not possible distinguish between the various components of a work-load condition (early perceptual, intermediate activity relevant, and late emotional components).

### SUMMARY OF THE INVENTION

The invention has as it's starting point the closest state of the art as described in the above-mentioned U.S. Pat. No. 5,884,626. The present invention is concerned with the task of developing an improved process and system for detecting the mental condition and work load values of a vehicle operator, which substantially overcomes the above-mentioned disadvantages of and, beyond this, exhibits further advantages.

In a process of the type set forth in the preamble of claim 1, this task is accomplished in accordance with the characterizing features of claim 1. The characterizing features of a device associated with the inventive process are described in claim 8. Further details of the invention and preferred various embodiments can be seen from the characterizing features of the dependent claims.

### BRIEF DESCRIPTION OF THE DRAWINGS

The inventive process and the corresponding device are described in the following on the basis of a preferred embodiment, wherein reference is made to the figures and the therein used reference numbers. There is shown:

FIG. 1 characteristic brain waves in the case of an epileptic attack;

FIG. 2 measurable changes of signals following stimulation.

### DETAILED DESCRIPTION OF THE INVENTION

The invention is based upon the fact that, besides fatigue and comfort, other influences can also detrimentally impair the ability of a person to operate. Various stages of the actual load condition and ability to process correlate with certain phases of brain activity, these are associated with changes in brain wave segments which are not limited to the α-band alone. Waves in lower and higher frequency bands can thus provide valuable supplemental information regarding the ability to work under pressure, receptiveness and effectiveness of a person. These also include impairment of operating ability due to illness or also medicaments or drugs.

In neurology in general, when making brain activity measurements, distinctions are made between the following various frequency bands:
the sub-δ-band with a frequency range of 0.15 to 0.5 Hz,
the δ-band with a frequency range of 0.5 to 3.5 Hz,
the θ-band with a frequency range of 3.5 to 8 Hz,
the α-band with a frequency range of 8 to 13 Hz,
the β-band with a frequency range of 13 to 30 Hz, as well as frequency components above the β-band (that is above 30 Hz).

Signals and their changes in the various bands can, in accordance with modern medical knowledge, be correlated with high reliability with various influences, mental reactions and impairments.

Besides the α-band already discussed in the state of the art, as carriers of information regarding the alertness condition of a person, frequency components above this band (β-band and above) provide information regarding impairment of mental functions due to drugs (for example, alcohol) or central acting medications (for example, cough suppressant with high codeine content). The ability to detect such impairments is of high importance in vehicle operating safety.

The θ-band, which is directly below and adjacent to the α-band, provides information of a different type, which however likewise has great importance for vehicle operating safety: depth of sleep phases correlate with significant changes in the signals in the θ-band. Spontaneously occurring sleep processes (short interval sleep) can occur in the case of high fatigue, and can however also occur in certain illnesses (so-called “narcolepsy”). By the continuous sensing or monitoring of this frequency area, the occurrence of a so-called “one second sleep” (frequently responsible for accidents) can be recognized relatively early and responded to by the triggering of a waking signal (acoustic, optical, tactile) or, in critical situations, in certain cases by direct intervention in the control of the vehicle (initiation of a braking process).

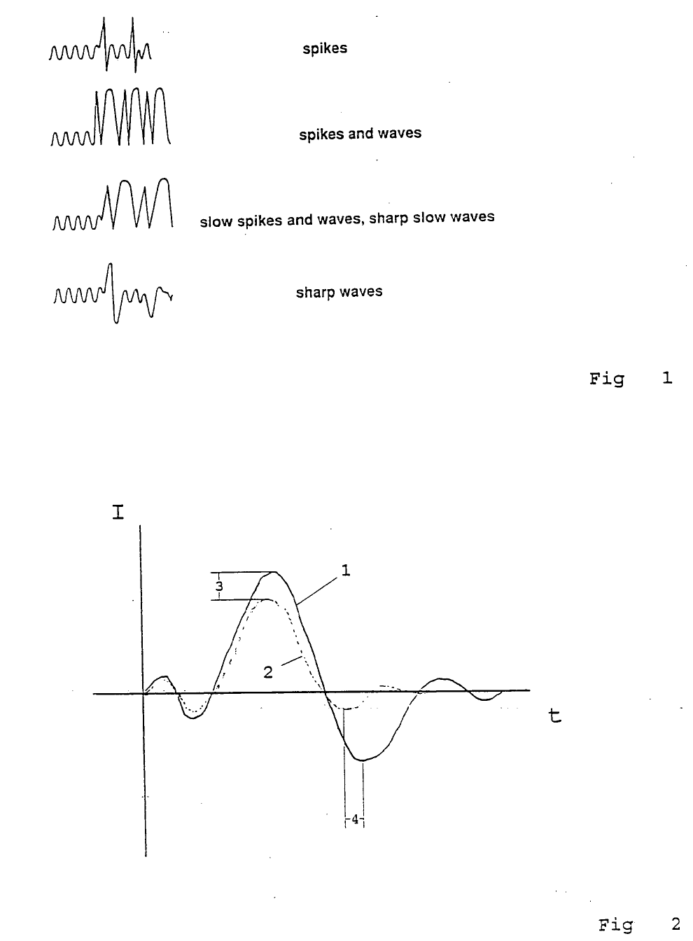

A different types of illness-dependent impairment of mental load ability or, as the case may be, impairment of ability to work under pressure, are, for example, epileptic attacks, which can occur spontaneously in various forms. These are always associated with characteristic changes in the brainwave activity, in particular changes in the δ-band and sub-δ-band. Typical signal shapes are shown in FIG. 1. One can distinguish various attack strengths, wherein however even a “light” attack in traffic can mean a high danger potential, since here interference in ability to understand already typically occurs. A so-called “aura” is the medical term for a short time and, as a rule, localized brain region impairment of function (focal” attack), which is associated with a significantly changed perception of reality and therewith strongly limits the vehicle operating proficiency. Also, the so-called “absences” occur spontaneously without subjectively noticeable advanced warning. In this type of attack any type of comprehension is completely interrupted for a time of typically less than 30 seconds, wherein this interruption of consciousness has an abrupt onset and likewise sudden end. Finally, a strong attack (grand mal) covers multiple regions of the brain simultaneously (“generalized” attack). One known trigger for this type of attack is pulsed light changes (flickering light). A condition of this type could occur while driving, for example, along a tree-lined lane, where a row of trees along the edge of the road can, when the sun is low to the horizon, produce this type of flickering light stimulus. A “precursor” of a generalized attack detectable in the EEG is synchronized signals.

In a further embodiment of the invention, stimuli are employed which trigger special mental activity. Suitable are, for example, optical patterns (for example pictograms, images, video sequences) and/or acoustic signals (for example sounds, speech, sequences of notes, music sequence) or however also tactile stimuli (for example moderate vibrations). These types of stimuli evoke brainwave potentials with characteristic progression characteristics. In FIG. 2 one example of an evoked potential is shown schematically. The signal (1) generated in the case of full alertness, changes when impairment occurs due to tiredness, over-exertion, medicaments, etc. By comparison of changes between normally evoked potential (=reference patterns) with an actually recorded signal (2), for example, with regard to amplitude height (3) and/or time displacement (4) of the curve maxima, indications regarding the current mental condition of the vehicle operator can be derived. It is known that a diminishment of the capacity to process information corresponds with changes of evoked potentials in the time range of 300 milliseconds (so called “P300”).

Influences which can impair the ability or capacity or, as the case may be, the receptiveness of the vehicle operator can be identified in accordance with the inventive process by an appropriately differentiated “multi-band” signal analysis, that is, over a very broad frequency range. The continuous sensing of the brainwave activity at multiple points of the cranial surface (for example using 32 electrodes) makes possible therein a mapping of or, as the case may be, area distribution analysis of the actual activity and association of individual components with various brain regions. In accordance with the invention signal evaluation includes in particular:
continuous monitoring of the intensity over time in the various bands,
identification of characteristic signal shapes and correlated signal changes (occurrence of synchronized signals or, as the case may be, signal changes at various sensing electrodes or as the case may be in various bands),
comparison of the detected signal shapes and changes with corresponding stored data (for example—reference patterns, fluctuations).

These evaluations can occur using conventional digital-electronic components, which are for example parts of a microprocessor system. A computer based system for analysis of EEG-patterns is described in, for example, U.S. Pat. No. 5,447,166.

In order to continuously monitor of the actual mental condition, ability to work under pressure, receptiveness and effectiveness of the vehicle operator during driving, brainwaves must be permanently recorded and analyzed for their neurological characteristic values. Conventional EEG-devices use electrodes which are in direct electrical contact with the scalp—for example via electrolyte-containing gels—for detecting the brainwaves to be evaluated. In the present case however there is a need for a certain degree of wear-comfort, in order not to interfere with operation of the vehicle. A light, wearable EEG-device is described for example in the periodical “Medical & Biological Engineering & Computing” 1994, page 459-461, H. Iguchi et al.: “Wearable Electroencephalogram System With Preamplified Electrodes”. Further refined systems with highly sensitive field detectors can likewise be employed. Therein the electrical and/or magnetic fields produced by the brainwaves are detected with contactless sensors and evaluated. Contactless EEG-measurements of this type have been carried out using, for example, SQUID-detectors. Such a device offers, depending upon the positioning of the field sensing detectors (for example in the area of the headrest) the advantage of a high comfort and accordingly high customer acceptance.

The inventive process and the inventive devices are particularly suited for employment in motor vehicles, since the necessary components (detectors, amplifiers, microprocessor systems, etc.) can be made very small and light in modern integration technology, and are also characterized by low energy consumption. The system is very flexible and can easily be adapted to various persons. Individual data for various vehicle operators can be stored, re-recorded, modified or erased. Likewise, the most various types of settings can be modified at will.

Basically, the inventive process and system contributes to safety in road traffic. If the effectiveness of the vehicle operator is compromised, then appropriate reactions for accident prevention can be introduced stepwise: from recommending a rest break at the onset of tiredness, to warning signals in the case of clear reduction in effectiveness, all the way to direct taking control of the vehicle in the case of critical impairment of perception or cognition.

1-10. (cancelled)

11. A process for monitoring the mental condition of a person while operating a vehicle by analysis of brainwaves, said process comprising:
sensing and analyzing brainwaves of the vehicle operator simultaneously in multiple frequency bands.

12. A process according to claim 11, wherein said frequency bands include two or more of the sub-θ-band, θ-band, δ-band, α-band, and β-band.

13. A process according to claim 11, wherein said brain waves are monitored for indicators of ability to work under pressure, receptiveness and effectiveness of the vehicle operator.

14. A process according to claim 11, wherein brainwaves are selectively analyzed for intensity changes in various frequency bands.

15. A process according to claim 11, wherein brainwaves are analyzed for characteristic signal shapes.

16. A process according to claim 11, wherein brainwave are analyzed in various frequency bands for characteristics which can be correlated with mental conditions.

17. A process according to claim 16, wherein said characteristics are synchronized signals or signal changes.

18. A process according to claim 11, wherein the brainwave analysis includes a comparison with stored data.

19. A process according to claim 18, wherein the stored data is in the form of reference patterns, significant values for correlations, or characteristic function parameters.

20. A process according to claim 11, further comprising generating a stimulus to stimulate the brainwaves.

21. A process according to claim 20, wherein the stimuli are visual, acoustic or tactile.

22. A process according to claim 11, further comprising:
analyzing the brainwaves of the vehicle operator for signal characteristics indicative of an impairment of the ability to work under pressure or diminished receptiveness or effectiveness,
upon detection of such signal characteristics, initiating a program including one or more graduated stepped up warning processes or control intervention processes.

23. A device for monitoring the mental condition of a person while operating a vehicle by analysis of brainwaves, said device comprising:
a sensor for sensing brainwaves and providing a signal to a microprocessor for analysis of said signals,
a microprocessor for storing and analysis of said signals simultaneously in multiple frequency bands.

24. A device according to claim 23, including
a program for analyzing the brainwaves of the vehicle operator for signal characteristics indicative of an impairment of the ability to work under pressure or diminished receptiveness or effectiveness, and
means for optical, acoustic and/or tactile stimulation of the person upon detection of such characteristics.

25. A device according to claim 23, wherein said sensor is a contactless field detector.
